(19) United States
(12) Patent Application Publication
(10) Pub. No.: US 2020/0054502 A1
(43) Pub. Date: Feb. 20, 2020

(54) MODIFIED BIO DEGRADABLE STRUCTURE FOR SINGLE USED HYGIENE PRODUCTS

(71) Applicants: Aariya WICKHAM, Perth, Western Australia (AU); Sylvia SENEVIRATNE, Perth, Western Australia (AU)

(72) Inventor: Aariya WICKHAM, Perth, Western Australia (AU)

(21) Appl. No.: 16/487,451

(22) PCT Filed: Feb. 21, 2017
(86) PCT No.: PCT/AU2017/050151

§ 371 Date: Aug. 21, 2019

(51) Int. Cl. A61F 13/514; A61F 13/53; D01F 2/06

(52) U.S. Cl. CPC A61F 13/51401; A61F 2013/530481; D01F 2/06; A61F 13/53

(57) ABSTRACT

The comfort of a newborn the feel the sensory is of importance in developing any type of clothing for a new born, infant to baby in terms of the 1st stich of clothing to mankind. We as specialist and scientist in natural fibres have researched and developed the most comfortable and healthy product to new born and babies. The feel, the comfort, the wearability in developing this product construction. The soft skin and the health and wellbeing of the baby has always been considered as the main priority in the present Invention in hope of addressing the ongoing infertility and skin irritation and skin dehydration on babies.

### HISTORY OF THE INVENTION

The comfort of a newborn the feel the sensory is of importance in developing any type of clothing for a new born, infant to baby in terms of the 1^{st} stich of clothing to mankind. We as specialist and scientist in natural fibres have researched and developed the most comfortable and healthy product to new born and babies. The feel, the comfort, the wearability in developing this product construction. The soft skin and the health and wellbeing of the baby has always been considered as the main priority in the present Invention in hope of addressing the ongoing infertility and skin irritation and skin dehydration on babies.

Nappy: The First Stitch of Clothing and Its Impact on Life; understand the importance of healthy development of babies through infancy to early childhood. The psychosocial development is in stages and consists of 8 stages. As to the requirement for the development of the first stich of clothing and to understand its impact on a new born to toddler is focused on this report with scientific reasoning and referenced by a psychoanalyst. The research will focus on the 1st stage; which is Hope: trust vs. Mistrust (oral-sensory, infancy, 0-2 years.) 2nd stage Will: autonomy vs. shame and doubt (early Childhood, 2-4 years.) A new born is also referred as a parasite clinging on to life of the mother or parents or guardian. Giving the most important emotional and physical love and care is the important factors for the wellbeing of a baby that will grow as a normal human at adult Sans. In manufacturing baby clothing, we consider the 3 norms which is Comfort, safety, and Hygiene. This is to assure the importance of feeling comforted and assuring health and safety for a baby that relies on an adult to feel safe and this is related to 1st stage which is Hope—Trust vs Mistrust (Sensory). For nine months the baby is developed in the most comforted and warm environment in the womb of their mother. Feeling very secure and trusted. The main reason for the first stich of clothing to be 100% safe and comfortable for the new born is to assure trust vs mistrust. This is the beginning of a life that could have adverse effects if the first feeling (sensory) the feel of the baby Nappy/Diaper that can result in many different physiological affects as an adult. Hope: trust vs mistrust (Oral—sensory, infancy, 0-2 years)•Existential Question: Can I trust this world? The first stage of Erick Erickson's theory centres around the infant's basic needs being met by the parents and this interaction leading to trust or mistrust. Trust defined by Ericson is “an essential truthfulness of others as well as fundamental sense of one's own trustworthiness. The infant depends on the parents, especially to the mother sustenance and comfort. This is where choices of the mother becomes paramount to fulfil her baby's fundamental needs which in this research is sensory that assures comfort. If the parent expose the child to comfort, warmth regularity and dependable affection, the infants view of the world will be on of trust. Should the parent fail to provide a secure environment and to meet the child's basic needs: a sense of mistrust will result. Development of mistrust can lead to feeling of frustration, suspicion, withdrawal, and lack of confidence. According to Erik Ericson, the major development task in infancy is to learn whether or not other people especially primary caregivers, regularly satisfy basic needs. If the caregivers are consistent sources of food, comfort, and affection, an infant learns trust-that others are dependable and reliable. It they are neglectful or perhaps abusive the infant instead learns mistrust—that the world he or she is born into is an undependable, unpredictable and possible a dangerous place. While negative, having some experience with mistrust allows the infant to gain an understanding of what constitutes dangerous situation later in life, yet being in the stage of Infant or toddler. A child's number one needs are to feel safe, comforted and well cared for. Virtues—WILL: autonomy vs. shame and doubt (early childhood, 2-4 years) As the child gains control over eliminative functions and motor abilities, they begin to explore their surroundings. The parents still provide a strong base of a security from which the child can venture out to assert their will. The parent's patience and encouragement helps foster autonomy in the child. Children at this stage like to explore the world around them and they are constantly learning about their environment. Caution must be taken at this age while children may explore things that are dangerous to their health and safety. Explanation of Somatosensory system (Somatosensory nervous system) The nerve cells that responds to changes to the surface or internal site of the body is a complex system which is also known as the sensory receptors. Nerve cells to the spinal cord where they may be processed by other nerve cells and then relayed to the brain for further processing. Sensory receptors are found in many parts of the body including the SKIN, epithelial tissues, skeletal muscles, bones and joints, internal organs and the cardiovascular system. The research would like to scientifically address the difference between fine touch and crude touch which our body understands and reacts and this would be the same for babies. Fine touch (feel) is a sensory modality Approximate Age Virtues Psychosocial Crisis Significant Relationship External Question Example Infancy 0-2 years HOPE Basic trust vs mistrust Mother Can I trust the world? Feeding, comfort, abandonment Early Childhood 2-4 years “WILL” Autonomy vs shame and doubt Parents Is it okay to be me? Toilet training, clothing themselves that allows a subject to sense and localize touch. The form of touch where localization is not possible is known as the crude touch. The posterior column-medial lemniscus pathway is the pathway responsible for the sensing of fine touch information to the cerebral cortex of the brain. Crude touch (Feel) is a sensory modality that allows the subject to sense that something has touched them, without being able to localize where they were touched (contrasting “fine touch). Its fibres are carried in the spinothalamic tract, unlike the fine touch, which is carried in the dorsal column. As fine touch (posterior column-medial lemniscus pathway) have been disrupted. Sensory is a crucial mean of receiving information. Eg; tack tile makings identify stairs for visually impaired people, Touch can result in many different physiological reactions such as a baby laugh at being tickled by an older. In the research we are now focussing the importance of the feel of the first stich of clothing and how it could have an impact on the new born—Infant. The feeling and comfort of the baby Nappy/diaper which is the first stich of clothing on human kind and the importance of the choices that have to be made to assure comfort and Trust. Having understood the physiological impact that could lead to antisocial behaviour in adult sands is what we are hoping to address in our research and the creation of a better choice assuring comfort to all babies. Natural air flow to body and natural material that would give the baby the sense of good feeling. Our researchers have been working on this subject for the past 10 years and realised that a change is needed. The disposable diaper has been around for the past 5 decades. The very first that was invented by Marion Donavan with parachute material and some tissue paper in 1948 began the disposable single use diaper Industry. Since then Proctor and Gamble and Kimberly clerk and many others came up with disposable diapers solely due to convenience and a post war scenario where women/mothers started to work and washing was not the best option. Understanding the convenience, companies started to develop single use nappies but may not have considered the extent of every detail which is comfort and safety as well as understanding the psychological impact the first stitch of clothing can have on a new born. Understanding this could effect a child's entire life. This brings us to the light of understanding the importance of a highly breathable, flexible, softest of the soft of materials that should be included in developing a baby diaper the first stich of clothing to mankind. Therefore in the detail description we have focused on giving detail information of each material which is the major components of the invention which are the highest quality and the most suitable and developed a product called the baby disposable nappy.

### DESCRIPTION OF THE INVENTION

In the constructed absorbency, the inventor developed the viscose fluff for higher absorbency through its manufacturing process and the use of high cross linked polymer (SAP) to minimise the artificial absorbency was the primary objective to develop a bio degradable and baby safe product. In this invention with the use of modified and developed cotton detailed in the description is to assure that it has been produced to ensure the comfort (sensory) and to allow natural air flow to the skin and body. Most of the components used is environmentally safe as they break down in a short period of time. This construction will be used in the single used diaper and other hygiene products which are baby disposable diapers, baby pads, breast pads and sanitary napkins for all ages.

The change to the absorbent structure and modified viscose, superabsorbent polymer and the Cotton is to reduce the health issues and infertility that is on the increase in western countries that use the highest percentage of single used diapers on babies. It was important to develop a product that is baby safe and to reduce the impact on the environment while mainly focusing on the sensory and its' effects to a baby and how this would affect in long term as an adult.

### Description of the Modified Layers

### Cotton Non Woven

In the present invention, we have used 100% cotton nonwoven back sheet of the diaper which allows natural air floor to the skin. The use of cotton in the present invention, cotton fibre properties most often considered in nonwoven applications are micronaire, length and strength. Neps may also be considered for applications where visual appearance is important. In the present invention, we used micronaire as of the nonwoven application.

Micronaire is an airflow measurement of fibre fineness. It is performed on a weighed test specimen, which is compressed to a specific volume in a chamber. Air is forced through the specimen and the resistance to the airflow is measured. This resistance is proportional to the linear density of the fibres (expressed in micrograms per inch), adjusted for the maturity of the fibre. If the exact linear density of the fibre needs to be determined, the maturity of the fibre must be determined by another measurement. On a typical year, the micronaire range for upland cotton is 3.0 to 5.5

Cotton fibre length varies genetically and any sample of cotton fibre shows an array, or distribution, of fibre length. The fibre length in the present invention as the mean length of the longer half of the fibres in the sample in hundredths of an inch. In the present invention, we have used 1.5 inches of fibres to develop the nonwoven, this is to assure comfort and soft feel to the baby.

To secure strength we have bundled fibres with ⅛ inch between the two sets of jaws, and measuring the force required to break the fibres. Results are reported as grams per tex or grams per denier. A “tex” is a unit equal to the weight in grams of 1,000 meters of fibre. Therefore, the strength reported is the force in grams required to break a bundle of fibres one tex unit in size.

### Viscose Fluff

In the present invention, we have used viscose fluff in the absorption and used a high percentage of the fluff to reduce the supper absorbent polymers considering the comfort and safety of the baby.

The appreciation of cellulose fibres in different fields of application like textiles, non woven and hygiene products is not only the fact that they are natural and have comfortable feeling, the main reason is their water absorbency which is much higher than the water absorbency of most synthetic non-cellulosic fibres. In the present invention, we have increased the absorbency for the use of baby diapers and other hygiene products and the viscose fibres have been functionalized to further increase their water absorbency. In the present invention, the applications where cellulosic fibres with lowered or even suppressed water absorbency that was required. Ways how to regulate the water absorbency of viscose fibre is of importance in the present invention into both directions. In the present invention, the structure of cellulose allows the storage of humidity inside the fibre which then is exchanged with the fibre environment giving a high wearer comfort by moisture regulation. It became obvious that the region for “absorbency” ends at around 400% where the hollow fibres still have a sufficient strength.

### Bio film—PLA

In the present invention, we have used PLA nonwoven fabric which is also called needle felted for the top sheet, and the cuff of the baby diaper. We also included PLA bio film to be the liquid impervious layer to maximise the bio degradability of the product. Due to the chiral nature of lactic acid, several distinct forms of is polylactide exist: poly-L-lactide (PLA) is the product resulting from polymerization of L,L-lactid. PLLA has a crystallinity of around 37%, a glass transition temperature 60-65° C., a melting temperature 173-178° C. and a tensile modulus 2.7-16 GPa. Heat-resistant PLA used in the present invention can withstand temperatures of 110° C. PLA is soluble in chlorinated solvents, hot benzene, tetrahydrofuran, and dioxan.

Polylactic acid can be processed like most thermoplastics into fibre for example, using conventional melt spinning processes and film. PLA has similar mechanical properties to PETE polymer, but has a significantly lower maximum continuous use temperature. The tensile strength for 3-D printed PLA was previously determined. It was found to range widely depending on printing conditions, which were obtained using RepRap 3-D printers.¹ Results of a recent study gave a printed tensile strength of around 50 MPa and show that the act of 3-D printing PLA affects its properties—they showed a strong relationship between tensile strength and percent crystallinity of a 3-D printed sample and a strong relationship between percent crystallinity and the extruder temperature.

The present invention is constructed by using 100% cotton nonwoven back sheet with the water proofing layer made of corn (PLA Nonwoven) as the liquid impervious layer. For absorbency, in the present invention we have modified the viscose fluff absorbency and used 95% viscose fluff and reduced the percentage of super absorbent polymer. In the present invention, we have increased the cuff for prevention of leaks and increased half centimetre than normal diapers. The Top sheet which is adjacent to the baby's skin in the present invention, is PLA 20 gms, nonwoven fabric. PLA which is derived from fermentation of Corn is a bio degradable substance. It will break down in landfill within a short period of time. The present invention, the inventor is conscious on the baby's health as well and softer to the environment. Our focus in the invention is to make sure babies don't suffer from trauma by using inferior quality materials in their first stich of clothing which is our primary objective mentioned at the beginning of this document supported by scientific evidence.

### Modified Cross Link Density Polymer and Viscose

Water-absorbing polymers, which are classified as hydrogels when cross-it absorb aqueous solutions through hydrogen bonding with water molecules. A SAP's ability to absorb water is a factor of the ionic concentration of the aqueous is solution. In deionized and distilled water, a SAP may absorb 300 times its weight (from 30 to 60 times its own volume) and can become up to 99.9% liquid, but when put into a 0.9% saline solution, the absorbency drops to maybe 50 times its weight. The presence of valence cations in the solution impedes the polymer's ability to bond with the water molecule.

In the present invention, the total absorbency and swelling capacity was controlled by the type and degree of cross-linkers used to make the gel. As of the safety of baby and to reduce health issues in our invention where we used high cross-link density polymers to exhibit lower absorbent capacity and swell, but the gel strength is firmer and can maintain particle shape even under modest pressure.

In the present invention, the viscose fibres have been functionalized to further increase their water absorbency. In the present invention, the applications where cellulosic fibres with lowered or even suppressed water absorbency are required. Ways how to regulate the water absorbency of viscose fibre is of importance in the present invention into both directions. In the present invention, the structure of cellulose allows the storage of humidity inside the fibre which then is exchanged with the fibre environment giving a high wearer comfort by moisture regulation. It became obvious that the region for “absorbency” ends at around 400% where the hollow fibres still have a sufficient strength for processing.

In the absorbency, the inventor developed the viscose fluff for higher absorbency through its manufacturing process and the use of high cross linked polymer (SAP) to minimise the artificial absorbency was our primary objective to develop a bio degradable and baby safe product. In the present invention, along with the crosslinked polymer and the viscose fluff with higher absorbency the inventor further modified cotton fibre detailed in the description to construct the layers to assure the product is baby safe and environmentally friendly.

1. A product accruing to the present invention is a special constructed absorbent core to eliminate the risk of nappy rash in babies due to the choice of fabrics and fibres that complement each other to maximize absorption naturally without dehydrating body moisture.

2. In the present invention as per claim 1 the outer layer is a cotton light weight nonwoven structure which allows natural air circulation to babies skin.

3. In the present invention as per claim 1 the liquid impervious layer is a film made out of corn which helps biodegradation while being environmentally friendly. As most nappies end up in landfill.

4. In the present invention as per claim 1 the configuration of the core by using natural viscose fluff that is high in absorption of any liquid due to the physical properties of this particular viscose fluff which has a higher absorbent capacity due to the method of construction of the fibre. The viscose is developed through the cellulose solution used to spin the rayon fiber, which may also be called viscose. This fibre is disbursed through a hi-tech machine in production where it breaks down to a fluff in the absorbent core of the baby diaper.

5. In the present invention as per claim 1 the supper absorbent polymer (SAP) is used in very minimal and is specially constructed for babies, this reduces the chemical order in the single use baby nappies and further reduces skin irritation in babies.

6. In the present invention as per claim 1, An EU Standard is in place for absorption under our manufacturing process.

7. In the present invention as per claim 1 due to the higher range of natural fibres the product has a high level of bio degradation which we claim 70% in 120 days which is a benefit to the environment that has been independently tested and certified.

8. In the present invention as per claim 1 due to the fibre construction in the absorbent core, the use of nappies in a baby per day is less whereby economical and supports the environmental benefits further which we address.

9. In the present invention as per claim 1 due to the supper soft bio film nonwoven used adjacent to skin, further prevent irritation to babies skin.
